(19) United States
(12) Patent Application Publication
(10) Pub. No.: US 2011/0243991 A1
(43) Pub. Date: Oct. 6, 2011

(54) PROCESS FOR PRODUCTION OF VACCINES

(75) Inventor: Jessica Reineke, Hofheim a T. (DE)

(73) Assignee: BOEHRINGER INGELHEIM VETMEDICA GMBH, Ingelheim am Rhein (DE)

(21) Appl. No.: 13/126,186

(22) PCT Filed: Dec. 1, 2009
(86) PCT No.: PCT/EP09/66109

§ 371 Date: Jun. 23, 2011

(30) Foreign Application Priority Data
Dec. 3, 2008 (EP) 08170591.5

(51) Int. Cl. A61K 39/08; C12P 21/06; A61K 39/02; A61P 37/04; A61P 31/04; A61P 1/12

(52) U.S. Cl. 424/239.1; 435/68.1; 424/236.1

(57) ABSTRACT

The invention relates to a process for the production of a vaccine against bacterial pathogens which produce an AB toxin, like Clostridium, comprising (a) culturing the pathogen under conditions where the AB toxin is produced, and harvesting the culture (b) cleaving the AB toxin enzymatically in vitro, preferably using inositol hexaphosphate as a co-factor, and (c) combining the composition of step (b) with a pharmaceutically acceptable carrier.

### TECHNICAL FIELD

The invention relates to a process of production of vaccines, and vaccines produced accordingly.

### BACKGROUND INFORMATION

Clostridium difficile, a spore-forming, gram-negative bacterium, is responsible for 60% of the cases of antibiotics-associated diarrhoea and for almost 100% of the patients affected by pseudomembranous colitis. The mechanism responsible for the outbreak of the disease is not yet fully understood. It might be related to both host and strain factors, since not all patients infected with C. difficile develop the disease. Clinical symptoms of infected patients can range from being asymptomatic to life-threatening toxic megacolon.

C. difficile, like many other pathogens causing disease in animals, including humans, produces toxins. A toxin is a poisonous substance produced by living cells or organisms that is active at very low concentrations. Toxins can be small molecules, peptides, or proteins and are capable of causing disease on contact or absorption with body tissues by interacting with biological macromolecules such as enzymes or cellular receptors. C. difficile produces two toxins, Toxin A (TcdA) and Toxin B (TcdB) which are causative for antibiotic-associated diarrhoea or pseudomembranous colitis. They are very large (308 kDa and 269 kDa) bacterial proteins, which are part of the family of the so-called large clostridial cytotoxins (LCT's) together with TcsH and TcsL of C. sordellii and Tcna of C. novyi. All these toxins display a high degree of sequence homology, a similar domain structure and harbour a glycosyltransferase moiety. TcdA and TcdB are single-chained proteins characterized by a tripartite functional organization. Their C-terminal domain is required for binding to the plasma membrane of the target cell, the hydrophobic middle part is a putative translocation domain and the N-terminal catalytic domain of the proteins carries the glycosyltransferase site. The uptake process into the cytosol of the target cell has not yet been fully characterized. However, it is generally accepted that the toxins are endocytosed after binding to cell surface receptors. After acidification of the endosomes, only the N-terminal domain of the toxin translocates into the cytosol. This translocation process is supposedly mediated by pore formation, since TcdA forms pores in artificial membranes at low pH. Activation of the toxin requires proteolytic cleavage between the amino acids Leu543 and Gly544, which liberates a small fragment of 63 kDa that harbours the N-terminal catalytic domain into the cytosol. The larger, 207 kDa C-terminal part of TcdB, remains in the membrane fraction. The N-terminal 63 kDa fragment displays full cytotoxic activity. Once liberated, the N-terminal glycosyltransferase domain can move freely in the cytosol to inactivate its target proteins, GTPases of the Rho/Rac family. These proteins are involved in many cellular functions, e.g. organization of the actin cytoskeleton, control of transcription, cell polarity and proliferation. Since Rho GTPases play an important role in many functions of the immune system, including pathogen defense responses, cytokine expression and signalling of immune cells, they constitute optimal targets for bacterial toxins.

It has recently been shown that activation of C. difficile toxins occurs by autocatalytic cleavage (Reineke et al., Nature 2007, 446:415-419). Furthermore, the role of inositol hexaphosphate (Ins6P, IP6, CAS number [83-86-3]) as a potent activator and co-factor of such autocatalytic cleavage of toxin B was elucidated (Reineke et al., supra). This highly charged molecule appears to fulfil several functions and might be involved in the stabilization of the conformation of the toxin. It is now known that toxin activation through autocatalytic cleavage occurs also with similar toxins of other organisms, including the LCT's toxin A (TcdA) and B (TcdB) of Clostridium difficile, the lethal (TcsL) and the hemorrhagic toxin (TcsH) of Clostridium sordellii, and the α-Toxin (Tcnα) of Clostridium novyi, the RTX toxins of Vibrio cholerae (VcRTX), V. vulnificus (VvRtx), V. splendidus (VsRtx), Xenorhabdus nematophila (XnRtx), X. bovienii (XbRtx), Yersinia pseudotuberculosis (YpRtx), Y. mollaretti (YmMfp2) and Bordetella pertussis (FhaL1-4) (Sheahan KL et al. EMBO J 2007, 26(10):2552-2561.) Listonella anguillarum, Photorhabdus luminescens, Aeromonas hydrophila and Yersinia enterocolitica (Lupardus P J et al. SCIENCE 2008, 322(5899):265-268). All these toxins can be classified as “AB toxins” as outlined below.

International patent application WO2008014733 discloses a method of treatment of Clostridium infection wherein an inhibitor or activator (IP6) of the autocatalytic activity is administered to a patient.

Several publications disclose C. difficile vaccines. Vaccines wherein TcdA and TcdB are incativated by the chemical agent formalin are disclosed in Sougioultzis S. L. et al., Gastroenterology (2005), 128:764-770; Kotloff, Infect. Immun. 2001; WO9920304, and Ghose et al, Infect. Immun. (2007), 75(6), 2826-2832. Vaccines comprising recombinantly expressed polypeptides representing the C-terminal ligand domain of TcdA or TcdB are disclosed in WO9859053, WO0061761, WO0061762, WO9702836, Pavliakova et al, Infect Immun (2000), 68(4), 2161-2166; Ward et al, Infect Immun (1999), 67(10), 5124-5132; and Lyerly et al., Current Microbiol 21: 29-32). WO2007146139 discloses a codon-optimised DNA-molecule encoding the receptor binding domain of TcdA and TcdB and is use as a DNA vaccine. WO2004041857 discloses non-toxic mutants of TcdB and its use for vaccination. Genth, H. et al., Infect. Immun. (2000), 68: 1094-1101 discloses a method to generate enzymatically deficient Clostridium difficile toxin B as an antigen for immunization.

Vaccines are typically manufactured by making a preparation comprising antigenic components of such pathogens and admixing them with a pharmaceutically acceptable carrier. To achieve an effective immune response and for economic reasons it is desirable to use preparations obtained from bacterial cultures with only few processing or fractionation steps. In the case of toxin producing organisms, the problem is that the toxins included in such preparations would prevent administration thereof unless they are inactivated. Therefore, the prior art approaches have suggested to make vaccines against bacterial pathogens producing AB toxins like C. difficile by chemical inactivation of the toxins, recombinant expression of the non-toxic domain of the toxins (the C-terminal receptor binding or “B” domain), or producing non-toxic mutants of the toxins. However, all these measures lead to a loss of antigenic epitopes of the pathogenic organism which may affect the effectivity of the vaccine, and/or are costly.

### BRIEF SUMMARY OF THE INVENTION

The present invention relates to a process for the production of a vaccine against bacterial pathogens which produce an AB toxin, comprising
(a) Culturing the pathogen under conditions where the AB toxin is produced, and harvesting the culture;
(b) Cleaving the AB toxin enzymatically in vitro; and
(c) Combining the composition of step (b) with a pharmaceutically acceptable carrier.

In a preferred aspect, the enzymatic cleavage is autocatalytic. In a preferred aspect, inositol phosphate, preferably inositol hexaphosphate is used as a co-factor of the enzymatic cleavage.

In a further preferred aspect, the invention relates to a process as described, wherein the cells are separated from the culture medium after the harvest, and the AB toxin in the culture medium is cleaved.

The process of the invention can be used for the production of vaccines against pathogens of the genus Clostridium, preferably C. difficile, C. sordellii, C. botulinum, C. perfringens, C. tetani, or C. novyi, or of the genus Vibrio, preferably V. cholerae, V. parahaemolyticus, V. vulnificus, or V. splendidus, or V. anguillarum or of the genus Xenorhabdus, preferably X. nematophila, X. bovienii, or of the genus Yersinia, preferably Y. pseudotuberculosis, Y. pestis, Y. enterocolitica, or Y. mollaretti, or of the genus Bordetella, preferably B. pertussis, B. parapertussis, or B. bronchiseptica, or of the genus Actinobacillus, preferably A. μleuropneumoniae, or A. suis and E. coli.

An adjuvant may be added to the vaccine composition.

In a further aspect, the present invention relates to a vaccine produced with the process as described.

Another aspect of the invention is related to the use of a vaccine produced according to the process as disclosed, for the vaccination of animals, including humans, against infection of bacterial pathogens producing AB toxins.

Another aspect of the invention relates to a method of vaccination of animals, including humans, against infection of bacterial pathogens producing AB toxins, comprising administering an effective amount of a vaccine produced according to the process of the invention to an animal, including a human.

### DETAILED DESCRIPTION OF THE INVENTION

The present invention relates to an improved process of production of vaccines against bacterial pathogens which produce toxins of the AB type (AB toxins). The present invention provides for an elegant method of inactivation of AB toxins by an autocatalytic enzymatic process in vitro taking advantage of the intrinsic proteolytic activity of such toxins. To this end, the composition containing the toxins is adjusted to conditions under which such enzymatic cleavage can occur. In particular, the addition of a necessary co-factor such as inositol phosphate can induce proteolytic inactivation of the AB toxin. By this proteolytic cleavage, the toxic A domain is separated from the transporter domain B and looses its ability to enter the cytosol of cells where it needs to be to exert its toxic effects. In effect, the resulting composition is not toxic any more, or much less toxic than the single chain AB toxin, when applied to living organisms. On the other hand, this kind of inactivation preserves the natural conformation and the antigenic epitopes of the proteins which is important for the effectivity of the vaccine.

In the context of the present invention, the term “AB toxin” is used for a single-chain bacterial toxin which, like the LCT's, comprises a catalytic domain (the A domain) and a receptor binding/translocation domain (the B domain or transporter domain), and wherein the activation of the catalytic domain in vivo occurs by autocatalytic cleavage releasing the catalytic domain into the cytosol. AB toxins are for example the LCT's including toxin A (TcdA) und B (TcdB) of Clostridium difficile, the lethal (TcsL) und the hemorrhagic toxin (TcsH) of Clostridium sordellii, and the α-Toxin (Tcnα) of Clostridium novyi. Furthermore, AB toxins include the RTX toxins of Vibrio cholerae (VcRTX), V. vulnificus (VvRtx), V. splendidus (VsRtx), Xenorhabdus nematophila (XnRtx), X. bovienii (XbRtx), Yersinia pseudotuberculosis (YpRtx), Y. mollaretti (YmMfp2), Y. enterocolitica (YST), Listonella anguillarum (VaRtx) and Bordetella pertussis (FhaL1-4).

Accordingly, the process of the invention may be applied to manufacture vaccines against infection by the bacterial pathogens producing AB toxins, as those bacteria listed above. A vaccine is a pharmaceutical preparation which is used to improve immunity to a particular disease in animal, including humans. Vaccines can be prophylactic (e.g. to prevent or ameliorate the effects of a future infection by any natural or “wild” pathogen), or therapeutic, i.e. applied in a situation where the host is already infected by the pathogen, with or without clinical symptoms of disease. Vaccines may contain killed micro organisms, modified live (attenuated) micro organisms, antigenic subunit preparations of micro organisms (e.g. fractions or recombinantly expressed polypeptides), or, as preferred in the context of the present invention, toxoids, i.e. inactivated toxic compounds in cases where these primarily cause the illness. The vaccine may contain an adjuvant, an agent that can stimulate the immune system and increase the response to a vaccine, without having any specific antigenic effect in itself. Examples of commonly used adjuvants are alum (hydrated aluminium potassium sulfate), aluminium phosphate, aluminium hydroxide, squalene, or oil-based adjuvants. A preferred adjuvant is the commercially available Carbopol® 934P (Carbomer 934P; Noveon, Inc. , Pedricktown, N.J., USA) which may be present in the amount of about 2 ml/l. Carbopol is an acrylic acid polymer which is cross-linked with polyallylsucrose.

The first step of the process is culturing the pathogen under conditions where the AB toxin is produced. Bacterial cell culture is well-established in the art. Standard methods for the different species are known and suitable samples of the microorganisms are available from public collections. Listed microorganisms are cultivated according to their special requirements. Clostridia are cultivated under anaerobic atmosphere whereas Yersinia, Xenorhabdus, Bordetella and Vibrio can be cultivated aerobically. Yersinia are psychrotolerant, these organisms are usually cultivated at 28° C. Each organism needs its special medium composition for growth which is readily known in the art.

The AB toxin is normally released into the culture medium in the late stationary phase of the culture. After the harvest, it is often advantageous to separate the cells from the culture medium, as the AB toxins are present in the medium in sufficient concentration. This can be done by centrifugation. When the cells are separated by centrifugation, they are discarded and the supernatant is further processed. The toxins in the medium are then deactivated by enzymatic cleavage taking advantage of their autocatalytic properties.

To achieve cleavage, the conditions have to be adjusted properly to allow for the enzymatic activity. Most importantly, a co-factor has to be added which promotes the enzymatic activity. Inositol phosophate, in particular inositol hexaphosphate, may be used as a co-factor at a concentration range of 1 μmol/l to 10 mmol/l, more preferably 10-100 μmol/l, but other analogues or derivates such as 1,3,4- or 3,4,6-triphopsphate, 1,2,3,4-, 1,3,4,5-, 3,4,5,6, or 1,4,5,6-tetraphosphate, or 1,2,3,4,5-, 1,2,3,5,6-, 1,3,4,5,6-, 2,3,4,5,6-pentaphosphate work as well, but may require higher concentrations. The pH should be in the range of 6,5-8,5, and the pH of the culture medium is normally already within that range. Otherwise, a buffer may be added or exchanged e.g. by dialysis or ultrafiltration, e.g. Tris HCl at pH 8.5. A suitable temperature range is 20-40° C. The cleavage will normally be completed within 1 to 24 hours and should be tested with an assay like those disclosed in the examples, to avoid residual toxicity.

The toxin species may be purified from the harvest and/or culture medium prior to inactivation. If the pathogen produces more than one toxin species, these toxin species may be isolated from each other before inactivation. For example, C. difficile produces the two toxins Toxin A (TcdA) and Toxin B (TcdB), as outlined above. These two proteins may be separated as exemplified in Example 4 herein before inactivation, and then used in vaccine preparation either individually, or in combination. Toxoid A and/or Toxoid B of C. difficile, i.e. Toxin A and/or Toxin B inactivated according to the present invention, are preferred antigens for vaccinations. The toxoids may be further purified after autocatalytic cleavage, with enrichment of the larger, C-terminal cleavage fragments (e.g. consisting of amino acids 543-2710 of the holotoxin A, or amino acids 544-2666 or 545-2666 of the holotoxin B). The purified toxoids of the invention may further be combined with inactivated AB toxins of other pathogens in vaccine preparations.

The resulting preparation is then brought into its final formulation for use as a vaccine as deemed appropriate. For example, it may be used as is, with the aqueous environment as is being the pharmaceutically acceptable carrier. The environment may also be changed, for example by dilution, dialysis, ultrafiltration or further purification steps like affinity chromatography. The antigen preparation may be freeze-dried for storage, for reconstitution with water before use. If appropriate, adjuvants may be added. Adjuvants that can be considered are for example water in oil-, oil in water-, multiphasic- or non-mineral oil- emulsions, aluminium based adjuvants, polymeric adjuvants like Carbopol®, squalene, liposome, microparticles, immunostimulatory complexes and Toll-like receptor cascade activating adjuvants. The vaccine will be administered by subcutan, interdermal, intramuscular, intravenous or intraperitoneal injection. The frequency of injection and dosage depends on the target species. Susceptible species are humans, dogs, cats, rabbits, pigs, cattle, fish, rodents and horses. The vaccination is a prophylactic treatment and protection of progeny can be achieved by vaccination of the mother. The time of vaccination starts after disappearance of maternal antibodies and may require booster vaccinations after 4 weeks and at later time points.

Thus, in a further aspect, the present invention relates to a vaccine against Clostridium-induced diarrhoea, comprising toxoid A and/or toxoid B of Clostridium difficile, wherein said toxoid A and/or toxoid B has been generated from Toxin A and/or Toxin B by autocatalytic cleavage, optionally together with a pharmaceutically acceptable carrier. In some embodiments, Toxoid A consists of amino acids 543-2710 of the sequences as deposited in public databases (EMBL, NCBI) under the accession Nos. YP_{—}001087137, ZP_{—}05349827, or YP_{—}003213641. In further embodiments, Toxoid B consists of amino acids 544-2666 or 545-2666 of the sequences as deposited in public databases (EMBL, NCBI) under the accession Nos. of YP_{—}001087135, ZP_{—}05349824, ZP_{—}05328744, YP_{—}003217086, or YP_{—}003213639. The vaccine may further comprise an adjuvant.

### EXAMPLES

### Example 1

### Manufacture of a Clostridium difficile Vaccine

Clostridium difficile samples may be obtained from public collections, for example the American Type Culture Collection (ATCC), Manassas, Va., USA, under accession No. ATCC 9689, ATCC 43255. It is grown in a fermenter in BHI medium (brain heart infusion broth, Becton Dickinson, Heidelberg, Germany; see American Pharmaceutical Association. 1950. The national formulary, 9th ed., APA, Washington, D.C.) under anaerobic conditions at 37° C. for 3-4 days. The two large cytotoxins TcdA and TcdB are released in the late stationary phase. At this point in time, the culture is harvested and the bacteria are sedimented by centrifugation at 8000×g for 10 minutes The supernatant is used as is, or toxins may be enriched by gel permeation chromatography (e.g. on S300 Sephacryl), affinity chromatography, anion exchange chromatography and/or ultrafiltration. The reducing agent dithiotreitol is then added at a final concentration of 1-50 mmol/l to the supernatant or toxin-enriched preparation, followed by addition of the chelate forming agent ethylene diamine tetraacetate at a final concentration of 10-100 mmol/l. Inositol hexaphosphate (IP6) is then added at a final concentration between 1 μmol/l and 10 mmol/l, for example 10 μmol/l or 100 μmol/l, and the composition is incubated at 37° C. for a period of 2 to 24 hours in a suitable buffer like Tris-HCL at a pH of 6,5-8,5. Completeness of the cleavage may be tested by sodium dodecylsulfate polyacrylamide gel electrophoresis (SDS-PAGE) and protein staining.

The resulting preparation is then brought into its final formulation for use as a vaccine as deemed appropriate. For example, it may be used as is, with the aqueous environment as is being the pharmaceutically acceptable carrier. The environment may also be changed,for example by dilution, dialysis ultrafiltration or further purification steps like affinity chromatography. If appropriate adjuvants may be added. Adjuvants that can be considered are for example water in oil-, oil in water-, multiphasic- or non-mineral oil-emulsions, aluminium based adjuvants, polymeric adjuvants like Carbopol, squalene, liposome, microparticles, immunostimulatory complexes and Toll-like receptor cascade activating adjuvants. The vaccine will be administered by subcutan, interdermal, intramuscular, intravenous or intraperitoneal injection. The frequency of injection and dosage depends on the target species. Susceptible species are humans, dogs, cats, rabbits, pigs, cattle and horses. The vaccination is a prophylactic treatment and protection of progeny can also be achieved by vaccination of the mother. The time of vaccination starts after disappearance of maternal antibodies and may require booster vaccinations after 4 weeks and at later time points.

### Example 2

### Activity Assay

CHO cells (chinese hamster ovary, e.g. DSM ACC110, Deutsche Sammlung von Mikroorganismen and Zellkulturen GmbH, Braunschweig, Germany) are seeded into 96 or 24 well microtiter plates (100 μl per well) in e.g. Ham's F10 medium with e.g. 5% FCS (fetal calf serum) and incubated at 37° C. overnight in a humid atmosphere until they reach confluence. They are washed with Ringer's solution without bivalent ions like Magnesium or Calcium, then the wells are filled with 100 μl (96 well μlate) or 400 μl (24 well μlate) Ringer's solution without Mg and Ca. Then, 100 or 400 μl, respectively, of the vaccine preparation of example 1, and respective dilution series (10⁻¹ to 10⁻⁸) are pipetted into the wells, with double values for each sample. BHI medium which was treated the same way as the vaccine preparation serves as a negative control. As a positive control, untreated C. difficile supernatant is used. The μlates are incubated at 37° C. in a humid atmosphere for 3 to 24 hours, then the cells are inspected with a microscope.

Cells treated with the vaccine preparation should show no morphological changes, as those of the negative control. Cells treated with untreated culture supernatant will show a cytopathic effect which is primarily characterized by getting a round shape and developing an “astrocyte-like” morphology. If the vaccine-treated cells show a cytopathic effect similar to the positive control, the enzymatic cleavage was not complete and has to be repeated.

### Example 3

### Vaccination of Animals

Syrian gold hamsters may be used as a standardized animal model for C. difficile infections. Animals of 60 to 100 g weight are used in the experiment. The animals receive different concentrations (1-100 μg) of vaccine preparation by intraperitonal or subcutaneous injection. The vaccine either contains no adjuvant, or Complete Freund adjuvant (1:1 with vaccine preparation) or Ribi (monophosphoryl lipid A and trehalose dicorynomycolate emulsion) as adjuvant. BHI medium treated the same way as the vaccine is used as a negative control. 2 weeks after the last vaccination, the animals receive 10-100 mg/kg clindamycin intraperitoneally or orogastrically. 24 hours later, they are inoculated through a gastric feeding tube or ball end cannula with at least 10⁴ viable C. difficile germs per animal, or 100 c.f.u. (colony forming units), respectively. Protective efficacy of the vaccine is determined through clinical monitoring of diarrhoea or mortality rate.

### Example 4

### Clostridium difficile Vaccination of Animals

In these studies, AB-toxins of Clostridium difficile were inactivated by use of their intrinsic autocatalytic cleavage function and used as vaccines in animals.

For toxin production, 1 ml of C. difficile (reference strain VP110463, ATCC 43255) working culture was transferred into a pretreated and sterile dialysis bag containing 200 ml 0.9% NaCl. The dialysis bag was μlaced into 1.3 I BHI medium and was incubated for 5 d at 37° C. in an anaerobic chamber. After 5 d the content of the dialysis bag was centrifuged (5000 rpm, 4° C., 15 min) and fractionated ammonium persulfate precipitation of the supernatant was performed. The first precipitation step (toxin A) was performed by addition of 45% (NH)₄SO₄ and stirring for 3 h at 4° C. After this time the solution was centrifuged (5000 rpm, 4° C. and 30 min) and a second precipitation step (toxin B) was conducted by addition of (NH)₄SO₄to a final content of 70%. The second fraction was stirred for 3 h at 4° C. and after that again centrifuged (5000 rpm, 4° C. and 30 min). The resulting pellets of the precipitation steps were suspended in 5 ml 50 mM Tris/HCl pH 7.5. The two fractions (toxin A and B) received from (NH)₄SO₄ precipitation were further purified by sucrose density gradient centrifugation. Therefore a sucrose density gradient was prepared by underlying 4.5 ml of the following sucrose solutions in increasing order in an ultracentrifuge tube: 10%, 18.75%, 27.50%, 36.25% and 45% sucrose in 50 mM Tris/HCl pH 7.5. The toxin fractions were added on the top and the tube was centrifuged for 3.5 h at 4° C. and 100.000 g. The resulting gradient was collected in 2 ml aliquots. The toxin content of the samples was measured by cytotoxicity assays with CHO-K1 cells. Therefore CHO-K1 cells (ATCC CCL-61) were grown in DMEM/F10-Medium containing 10% FCS, 0.5% L-glutamine and 0.5% penicillin/streptomycin. Monolayers of cells (about 4000 per well) were prepared in 96-well microtiter μlates and incubated at 37° C. and 5% CO₂ for 24 h. After incubation time 10- fold dilutions of toxin containing samples were prepared. After removal of medium from cells, toxin dilutions were added. Cytotoxic effects were examined microscopically after 24 h by an inverted microscope.
Following scheme was used to evaluate cytotoxicity:
positive (+): >90% rounded cells
negative (−): <90% rounded cells

The toxin containing fractions of the sucrose density gradient centrifugation were pooled (each for Toxin A and Toxin B) and 1:2 diluted with 50 mM Tris/HCl pH 7.5. The samples (either Toxin A or Toxin B) were loaded on an ion exchange column and a NaCl gradient ranging from 50 mM NaCl in 50 mM Tris/HCl pH 7.5 to 700 mM NaCl in 50 mM Tris/HCl pH 7.5 was conducted (Δ NaCl 5 mM/ml) to elute the toxin. Fractions of 2 ml were collected. Toxin content of the samples was measured by cytotoxicity assays with CHO-K1 cells. The toxin containing fractions were pooled and concentrated by an ultra centrifugation step for 15 min at 4° C. and 5000 rpm. To the obtained toxin solutions 20% glycerin was added and samples were stored at −20° C.

Purification steps were also monitored by SDS-PAGE. Proteins were visualized by Coomassie staining. Concentration of the final toxin samples were determined by comparison of the toxin amount in SDS gels with BSA standards. The comparison was conducted by optical adjustment and by computer analysis.

Vaccine preparations were prepared by induction of autocatalytic cleavage of toxins by adding DTT and IP₆. Toxin A cleavage was performed in H₂O at a final volume of 50 μl by addition of 3 mM IP₆ and 50 mM DTT. Toxin B cleavage was performed in H₂O at a final volume of 100 μl by addition of 1 mM IP₆ and 150 mM DTT. Autocleavage was performed over night at 37° C. on a rotator.

The resulting cleavage products were analyzed by cytotoxicity assay and SDS PAGE analysis. Cytotoxicity assays were performed with CHO-K1 cells and Caco cells as Caco cells showed higher sensitivity against Toxin A. Caco cells were grown in MEM medium containing 10% FCS and 0.5% penicillin/streptomycin on 96 microtiter wells incubated at 37° C. and 5% CO₂ for 48 h. The cytotoxicity assay showed a reduction of cytotoxicity of at least 10³ fold for toxin A and 10⁴ fold for toxin B after 24 h. Cleavage efficiency was visualized with Coomassie stained SDS PAGE analysis.

Vaccine doses with respective inactivated toxins (toxoids) were prepared with the Sigma Adjuvant System (oil-in-water emulsion with Monophosphoryl Lipid A and synthetic trehalose dicorynomycolate). Toxoid concentrations were determined by comparison of the toxin amount in SDS gels with BSA standards. The comparison was conducted by optical adjustment and by computer analysis. The adjuvant was reconstituted as described by the manufacturer with PBS and mixed 1:1 with the respective toxoid sample.

The classical model organism for Clostridium difficile infection is the Syrian hamster. Syrian hamsters react very sensitive to the infection and develop clinical signs and pathological alterations similar to those in humans. Thus the hamster infection model is a very strict model ending in a 100% mortality of infected animals.

In the trials Syrian hamsters with a body weight of 60-80g at inclusion were purchased by Charles River, D-97633, Sulzfeld, Germany. The animals were randomized to the respective groups at the arrival at the test facility and were given an adequate acclimatization period of at least 5 days. Animals were vaccinated 3 times in intervals of 2 weeks. 14 days after the last immunization the infection with Clostridium difficile was performed. 24 h prior to the oral challenge with Clostridium difficile, 2mg Clindamycin was orally administered to each animal. The administration of the antibiotic Clindamycin predisposes the animals to the C. difficile infection by disrupting the normal bacterial flora of the gut.

Over a period of 7 days after challenge careful daily clinical examinations were made and clinical findings recorded. At several time points during the study blood samples were taken and sera analyzed for antibodies that inhibit the cytotoxicity of Toxin A and Toxin B on cultured cells. Therefore CHO-K1 cells were seeded at 5000 cells/well in 96 well μlates in DMEM/F10-mdeium containing 10% FCS, 0,5% L-glutamine and 0,5% penicillin/streptomycin and were incubated at 37° C. and 5% CO₂ overnight. Dilutions of hamster sera were prepared in medium and incubated for 1 h at 37° C. with toxin A and B dilutions. The toxins had been diluted to concentrations that cause >90% rounding of cells after 3 h and 24 h. Cell rounding was determined microscopically after 3 h and 24 h as described before. Neutralization titer was defined as the reciprocal value of the greatest sera dilution which completely inhibited rounding of cells after 24 h.

The objective of the study was to determine if the repeated subcutaneous immunizations with different doses of a combination of inactivated toxin A and B is biocompatible and if protection against a C. difficile infection can be induced. Therefore 12 male Syrian hamsters were used that were randomized to 4 respective groups at the arrival at the test facility. Each group consisted of 3 animals. The groups were vaccinated on different days and increasing doses in order to be able to react to potential toxic effects after vaccination.

| | Group 1 | Group 2 | Group 3 | Group 4 |
|---|---|---|---|---|
| N (animals) | 3 | 3 | 3 | 3 |
| Dose volume | 100 μl Sigma Adjuvant System per dose | | | |
| 1. Immunisation | Toxoid A/B, | Toxoid A/B, | Toxoid A/B, | Adjuvant |
| | each 1 μg | each 1 μg | each 1.5 μg | |
| 2. Immunisation | Toxoid A/B, | Toxoid A/B, | Toxoid A/B, | Adjuvant |
| | each 2 μg | each 3 μg | each 4 μg | |
| 3. Immunisation | Toxoid A/B, | Toxoid A/B, | Toxoid A/B, | Adjuvant |
| | each 4 μg | each 4 μg | each 4 μg | |

study outline:

| Day of | |
|---|---|
| the | |
| study | Procedure |
| −5 | Baseline blood samplings, approximately 100 μL serum per |
| | animal |
| 0 | Administration of Group A and D with the respective test item, |
| | subcutaneous, 100 μL and body weight determination |
| 2 | Administration of Group B with the respective test item, |
| | subcutaneous, 100 μL and body weight determination |
| 4 | Administration of Group C with the respective test item, |
| | subcutaneous, 100 μL and body weight determination |
| 14 | Administration of Group A and D with the respective test item, |
| | subcutaneous, 100 μL and body weight determination |
| 16 | Administration of Group B with the respective test item, |
| | subcutaneous, 100 μL and body weight determination |
| 18 | Administration of Group C with the respective test item, |
| | subcutaneous, 100 μL and body weight determination |
| 30 | Administration of group A-D with the respective test item, |
| | subcutaneous, 100 μL and body weight determination |
| 38 | Blood sampling, approximately 100 μL serum per animal |
| 43 | Treatment with antibiotic (Clindamycin 2 mg/animal), orally |
| 44 | Challenge with Clostridium difficile (1.77 × 10⁸/animal) orally, |
| | 24 hours after treatment with antibiotic; body weight |
| | determination |
| 45-47 | Clinical adspection 5 times per day |
| 48-51 | Clinical adspection 3 times per day |
| 52 | Blood samplings of surviving animals |
| | Section of animals- on day 52, in case of death or at day of |
| | euthanizing (if animals developed severe signs of suffering). |

All animals were without clinical signs after subcutaneous vaccination thus the toxin dose could be increased to up to 4 μg each for toxoid A and B. Also the animals of the control group showed no reactions to the subcutaneous vaccination demonstrating that the Sigma adjuvant system was well tolerated if applied subcutaneously. The determination of the body weight at several days during the study also confirmed the good tolerance of the vaccination. All animals gained weight until challenge. The weight gain of the toxin vaccinated animals was comparable to that of the control group. Also the last measured weight before challenge of toxin vaccinated animals (average: Group 1: 140,3 g; Group 2: 136,3 g; Group 3: 144 g) was in the range of the weight of control animals (average Group 4: 141 g).

After the challenge at study day 44 the animals of the control group died within 2-3 days. In comparison the animals vaccinated with the cleaved toxins all survived longer and 2 animals even showed nearly no clinical signs until the end of the study (Group 1 animal No.1; Group 3 animal No.1).

Animal No. 1 from group 1 only developed softish feces at study day 47 but recovered fast and was without clinical findings at the next study day and until end of the study. The other animal that survived until end of study (No. 1 group 3) showed softish feces, slightly wet, soiled perineum and slightly reduced spontaneous activity from study day 47 onwards until study day 50. It was without clinical signs on study day 51.

| | Animal | Death/Euthanasia at |
|---|---|---|
| Group | No. | study day |
| Group 1 | 1 | study end (52) |
| | 2 | 48 |
| | 3 | 50 |
| Group 2 | 1 | 48 |
| | 2 | 48 |
| | 3 | 48 |
| Group 3 | 1 | study end (52) |
| | 2 | 48 |
| | 3 | 50 |
| Group 4 | 1 | 46 |
| (control group) | 2 | 47 |
| | 3 | 47 |

Based on these results, the immunizations with toxoid NB have induced immunogenicity and partial protection: All vaccinated animals survived longer than the control animals. While challenge was lethal for all control animals 2/9 vaccinated animals survived until the end of the trial. Furthermore the analysis of hamster sera in the cytotoxicity neutralization assay confirmed the development of an immunological response.

| | | Serum cytotoxicity | | Serum cytotoxicity | |
|---|---|---|---|---|---|
| Group | | neutralization titer | | neutralization titer | |
| (No. of | | d = −5 | | d = 38 | |
| hamsters) | Animal | Anti-A | Anti-B | Anti-A | Anti-B |
| 1 (3) | 1 | 0 | 0 | 50 | ≦10 |
| | 2 | 0 | 0 | 25 | 10 |
| | 3 | 0 | 0 | 50 | ≦10 |
| 2 (3) | 1 | 0 | 0 | 25 | ≦10 |
| | 2 | 0 | 0 | 50 | ≦10 |
| | 3 | 0 | 0 | 50 | ≦10 |
| 3 (3) | 1 | 0 | 0 | 10 | 10 |
| | 2 | 0 | 0 | 50 | ≦10 |
| | 3 | 0 | 0 | 50 | ≦10 |
| 4 (3) | 1-3 | 0 | 0 | 0 | 0 |

No cytotoxicity neutralizing antibodies against toxin A or B could be detected in the sera prior to vaccination and animals of the control group stayed without neutralizing antibodies until the last blood sampling at study day 38. All vaccinated animals developed neutralizing antibodies against both toxins after the three vaccinations (study day 38) clearly showing the immunogenicity of the inactivated toxin preparation. Also the challenge titer might be too high in this sensitive hamster model and might need further adaptation. Looking at the survival rates of the different vaccine groups, no dose-related effect could be observed. This can correlate to the small differences in toxoid concentration applied and therefore all vaccinated animals may rather be considered as one vaccine group.

Taken together, the results of this study demonstrate that by autocatalytic cleavage inactivated toxin is well tolerated, can induce an immunological response and partial protection against the infection.

1. Process for the production of a vaccine against bacterial pathogens which produce an AB toxin, comprising
a) Culturing the pathogen under conditions where the AB toxin is produced, and harvesting the culture;
b) Cleaving the AB toxin enzymatically in vitro; and
c) Combining the composition of step (b) with a pharmaceutically acceptable carrier.

2-12. (canceled)

13. Process of claim 1, wherein inositol phosphate is used as a co-factor of the enzymatic cleavage.

14. Process of claim 13, wherein said inositol phosphate is inositol hexaphosphate.

15. Process of claim 1, wherein the cells are separated from the culture medium after the harvest, and the AB toxin in the culture medium is cleaved.

16. Process of claim 1, wherein the AB toxin is purified from the harvest before cleaving.

17. Process of claim 16, wherein the AB toxin is harvested from the culture medium.

18. Process of claim 1, wherein the AB toxin is harvested from the culture medium.

19. Process of claim 1, wherein the pathogen is of the genus Clostridium.

20. Process of claim 19, wherein the pathogen is C. difficile, C. sordellii, C. botulinum, C. perfringens, C. tetani, or C. novyi.

21. Process of claim 1, wherein the pathogen is of the genus Vibrio.

22. Process of claim 21, wherein the pathogen is V. cholerae, V. parahaemolyticus, V. vulnificus, V. splendidus or V. anguillarum.

23. Process of claim 1, wherein the pathogen is of the genus Xenorhabdus.

24. Process of claim 23, wherein the pathogen is X. nematophila or X. bovienii.

25. Process of claim 1, wherein the pathogen is of the genus Yersinia.

26. Process of claim 25, wherein the pathogen is Y. pseudotuberculosis, Y. pestis, Y. enterocolitica, or Y. mollaretti.

27. Process of claim 1, wherein the pathogen of the genus Bordetella.

28. Process of claim 27, wherein the pathogen is B. pertussis, B. parapertussis, or B. bronchiseptica.

29. Process of claim 1, wherein the pathogen is of the genus Actinobacillus.

30. Process of claim 29, wherein the pathogen is A. pleuropneumoniae, or A. suis.

31. Process of claim 1, wherein the pathogen is E. coli.

32. Process of claim 20, wherein the pathogen is Clostridium difficile, and Toxin A is purified from Toxin B before cleaving.

33. Process of claim 1, wherein an adjuvant to added to the composition.

34. Vaccine produced with the process according to claim 1.

35. Vaccine produced with the process according to claim 32.

36. The vaccine of claim 32 comprising Toxoid A and/or Toxoid B of Clostridium difficile.

37. Vaccine against Clostridium-induced diarrhea, comprising Toxoid A and/or Toxoid B of Clostridium difficile, wherein said Toxoid A and/or Toxoid B has been generated from Toxin A and/or Toxin B by autocatalytic cleavage.

38. Vaccine according to clam 37, wherein said vaccine has a pharmaceutically acceptable carrier.

39. Use of a vaccine produced according to claim 1 for the vaccination of animals against infection of pathogens producing AB toxins.

40. Method of vaccination of animals against infection of pathogens producing AB toxins, comprising administering an effective amount of a vaccine produced according to claim 1 to an animal.
